(19) United States
(12) Patent Application Publication
(10) Pub. No.: US 2018/0142924 A1
(43) Pub. Date: May 24, 2018

(54) PROTECTIVE VEST APPARATUS AND SYSTEM

(71) Applicant: Eddy Limon, Long Beach, CA (US)

(72) Inventor: Eddy Limon, Long Beach, CA (US)

(21) Appl. No.: 15/572,159

(22) PCT Filed: May 9, 2016
(86) PCT No.: PCT/US16/31545

§ 371 Date: Nov. 6, 2017

Related U.S. Application Data
(60) Provisional application No. 62/159,156, filed on May 8, 2015.

(51) Int. Cl. F25B 21/02; F41H 1/02; G01S 19/13; A41D 13/002; A41D 13/005

(52) U.S. Cl. CPC F25B 21/02; F41H 1/02; G01S 19/13; A61F 2007/0234; A41D 13/0053; F25B 2321/0251; F25B 2321/0211; A41D 13/0025

(57) ABSTRACT

A protective vest and associated system are described. The vest may include at least one cooling subsystem configured to cool the torso of the wearer. The cooling subsystem may employ a fan, a coolant bladder with non-toxic coolant, a thermoelectric module and a heat sink. A heat exchange aperture in the vest shell may be enveloped by an armor shroud which also forms a fan air flow duct. The vest may include temperature sensors and a heart rate monitor, and may communicate with a wearer computing device by which the vest may be controlled and the geographical position of the vest and condition of the wearer may be monitored. The wearer computing device may further provide real-time tactical information and vest performance data to the wearer. Multiple wearer computing devices corresponding to respective protective vests may communicate with each other, or a department computing device, by way of a server.

### RELATED APPLICATIONS

This application claims the benefit of U.S. Provisional Application No. 62/159,156 filed May 8, 2015, the content of which is incorporated by this reference its entirety for all purposes as if fully set forth herein.

### TECHNICAL FIELD

The present invention relates generally to garments for protecting the wearer. More particularly, the present invention relates to a protective vest, such as a bullet-resistant vest, which provides cooling and other ancillary features.

### BACKGROUND

What is needed in the art of protective vests is the ability to more efficiently cool the wearer in a readily controlled or selectable manner. Moreover, it would be desirable to provide a system incorporating a protective vest that provides the wearer and their supervisors with improved situational awareness, access to information and tactical advantage.

### SUMMARY

A protective vest may preferably comprise a vest shell, at least one cooling subsystem and an onboard power supply. The vest shell may include an outer layer, an inner layer and a ballistic armor layer (such as Kevlar® or the like) disposed therebetween. Each cooling subsystem may include a cooling disbursement element, a thermoelectric module and a heat dissipation assembly. The cooling disbursement element may comprise at least one coolant bladder attached to the inner layer. The heat dissipation assembly may include a fan element and a heat sink. The thermoelectric module may be in heat-transferring communication between the cooling disbursement element and the heat dissipation assembly. The fan element may preferably be configured to force a flow of air across the heat sink. The power supply may be in electrical power-providing communication with the thermoelectric module and the fan element.

The vest shell may have a heat exchange aperture extending from the inner layer of the vest shell through the outer layer. At least a portion of either the heat sink or the thermoelectric module, or the combined heat sink and thermoelectric module, may protrude through the heat exchange aperture such that at least a portion of the fins of the heat sink extend outwardly of the outer layer.

A protective vest system may comprise one or more protective vests and respective wearer computing devices (such as a smartphone or the like). The wearer computing device may be in network communication with the protective vest for sending vest commands to the protective vest and receiving vest information from the protective vest. The system may further comprise a department computing device in network communication with the wearer computing device. Moreover, the network communication between the department computing device and the wearer computing device may be way of a server element.

The wearer computing device may be configured to command (e.g., by way of a wearer software application) the speed of the fan elements and the voltage applied to the thermoelectric modules. The protective vest may include a heart monitor and at least one temperature sensor. In such cases, the wearer computing device may be configured to receive (e.g., by way of the wearer software application) data from the protective vest based on the measurements of the heart rate monitor and temperature sensors.

The protective vest may include a GPS subsystem. In such embodiments, the wearer computing device may receive data from the protective vest based on positioning data of the GPS subsystem. The protective vest may include an onboard power supply, and the wearer computing device may receive data from the protective vest concerning the performance of the power supply.

### BRIEF DESCRIPTION OF THE DRAWINGS

Further advantages of the present invention may become apparent to those skilled in the art with the benefit of the following detailed description of the preferred embodiments and upon reference to the accompanying drawings in which:

FIG. 1 is a diagrammatic perspective view of one example of a protective vest in accordance with the present invention, shown deployed on the torso of a wearer;

FIG. 2 is a diagrammatic rear view of the protective vest shown in FIG. 1, and illustrating an example rear heat dissipation assembly in one example orientation;

FIG. 3 is a diagrammatic rear view similar to that of FIG. 2, but illustrating the example rear heat dissipation assembly in an alternative orientation, thus reducing its vertical profile;

FIG. 4 is a diagrammatic partial perspective view of an example rear heat dissipation assembly, and an example of the airflow produced thereby;

FIG. 5 is a diagrammatic partial perspective view of one example heat dissipation assembly, illustrating the fan element attached to the outer layer of a shell by way of stitching or strapping;

FIG. 6 is a diagrammatic cross-sectional view of one example cooling subsystem integrated with the front or rear portion of a vest shell;

FIG. 7 is a diagrammatic cross-sectional view taken orthogonally to the view in FIG. 6;

FIG. 8 is a diagrammatic cross-sectional view similar to that of FIG. 7, but wherein the protective vest includes an example armor shroud element secured to the vest shell and extending across the heat sink so as to define a heat sink air flow duct and protectively envelop the heat exchange aperture;

FIG. 9 is a diagrammatic rear view similar to that of FIG. 2, but wherein the vest includes an armor shroud element of FIG. 8;

FIG. 10 is a diagrammatic view of one example of a protective vest system in accordance with one non-limiting embodiment of the present invention, including computer and server elements in network communication with the protective vest;

FIG. 11 is a schematic diagram of one example electrical circuit onboard the protective vest, wherein the degree of cooling is manually or automatically controllable by way of a potentiometer or the like;

FIG. 12 is a diagrammatic exploded view of an example fan element and an example housing therefor;

FIG. 13 is a diagrammatic assembled view of the fan element and housing of FIG. 12, whereby the housing protects the axial inlet of the fan element from obstruction and allows air to be received from both an axial and radial direction, and the fan output airflow is directed by the nozzle of the housing;

FIG. 14 is a diagrammatic wireframe view of a login screen on one example wearer software application operating on a wearer computing device as part of an example protective vest system;

FIG. 15 is a diagrammatic wireframe view of an example home screen of a wearer software application, showing respective selectable screen navigation options;

FIG. 16 is a diagrammatic wireframe view of an example continued home screen of a wearer software application, showing respective selectable screen navigation options;

FIG. 17 is a diagrammatic wireframe view of an example Tactic/Traffic/Crime screen of a wearer software application, showing respective selectable screen navigation options;

FIG. 18 is a diagrammatic wireframe view of an example Tactics screen of a wearer software application, showing respective viewable or selectable options to enter or receive data;

FIG. 19 is a diagrammatic wireframe view of an example Shootings screen of a wearer software application, showing respective viewable selectable options to enter or receive data;

FIG. 20 is a diagrammatic wireframe view of an example Major Police Shootings screen of a wearer software application, showing respective viewable or selectable options to enter or receive data;

FIG. 21 is a diagrammatic wireframe view of an example Terrorist Attacks screen of a wearer software application, showing respective selectable options to enter or receive data;

FIG. 22 is a diagrammatic wireframe view of an example Traffic Alerts screen of a wearer software application, showing respective viewable or selectable options to receive data;

FIG. 23 is a diagrammatic wireframe view of an example Crime Alerts screen of a wearer software application, showing respective viewable or selectable options to receive data;

FIG. 24 is a diagrammatic wireframe view of an example Email screen of a wearer software application, showing respective viewable or selectable options to enter or receive data;

FIG. 25 is a diagrammatic wireframe view of an example Messages screen of a wearer software application, showing respective viewable or selectable options to enter or receive data;

FIG. 26 is a diagrammatic wireframe view of an example Special Orders screen of a wearer software application, showing respective viewable or selectable options to enter or receive data;

FIG. 27 is a diagrammatic wireframe view of an example Subpoena Control screen of a wearer software application, showing respective viewable or selectable options to enter or receive data;

FIG. 28 is a diagrammatic wireframe view of an example Phone screen of a wearer software application, showing respective viewable or selectable options to enter or receive data;

FIG. 29 is a diagrammatic wireframe view of an example Maintenance/Updates screen of a wearer software application, showing respective viewable or selectable options to enter or receive data;

FIG. 30 is a diagrammatic wireframe view of an example Vest screen of a wearer software application, showing respective viewable or selectable options to enter or receive data sent to or provided by the wearer's protective vest;

FIG. 31 is a diagrammatic wireframe view of an example GPS screen of a wearer software application, showing respective viewable or selectable options to enter or receive data sent to or provided by the wearer's protective vest;

FIG. 32 is a diagrammatic wireframe view of an example Vitals screen of a wearer software application, showing respective viewable or selectable options to enter or receive data sent to or provided by the wearer's protective vest;

FIG. 33 is a diagrammatic wireframe view of an example Copware® screen of a wearer software application, providing access to the third-party Copware® service;

FIG. 34 is a diagrammatic wireframe view of an example Department/Military Phonebook screen of a wearer software application, showing respective viewable or selectable options to enter or receive data;

FIG. 35 is a diagrammatic wireframe view of an example Traffic Codes screen of a wearer software application, showing respective viewable or selectable options to enter or receive data;

FIG. 36 is a diagrammatic wireframe view of an example Juvenile Codes screen provided by way of a wearer software application, showing respective viewable or selectable options to enter or receive data;

FIG. 37 is a diagrammatic wireframe view of an example Parking Codes screen provided by way of a wearer software application, showing respective viewable or selectable options to enter or receive data;

FIG. 38 is a diagrammatic wireframe view of an example Booking Bail Amounts screen provided by way of a wearer software application, showing respective viewable or selectable options to receive data;

FIG. 39 is a diagrammatic wireframe view of an example Traffic and RFC Cite Dates screen provided by way of a wearer software application, showing respective viewable or selectable options to receive data;

FIG. 40 is a diagrammatic wireframe view of an example Police/Military Manual screen provided by way of a wearer software application, showing respective viewable or selectable options to receive data;

FIG. 41 is a diagrammatic wireframe view of an example Detectives Manual screen provided by way of a wearer software application, showing respective viewable or selectable options to receive data;

FIG. 42 is a diagrammatic wireframe view of an example Employee Data screen provided by way of a wearer software application, showing respective viewable or selectable options to receive data relating to the current wearer of the corresponding protective vest;

FIG. 43 is a diagrammatic wireframe view of an example Videos screen provided by way of a wearer software application, showing respective viewable or selectable options to enter or receive data; and

FIG. 44 is a diagrammatic wireframe view of an example Flashlight screen provided by way of a wearer software application, showing respective viewable or selectable options to receive data pertaining to the wearer's flashlight.

### DETAILED DESCRIPTION OF THE PREFERRED EMBODIMENTS

Referring now to the drawings, like reference numerals designate identical or corresponding features throughout the several views.

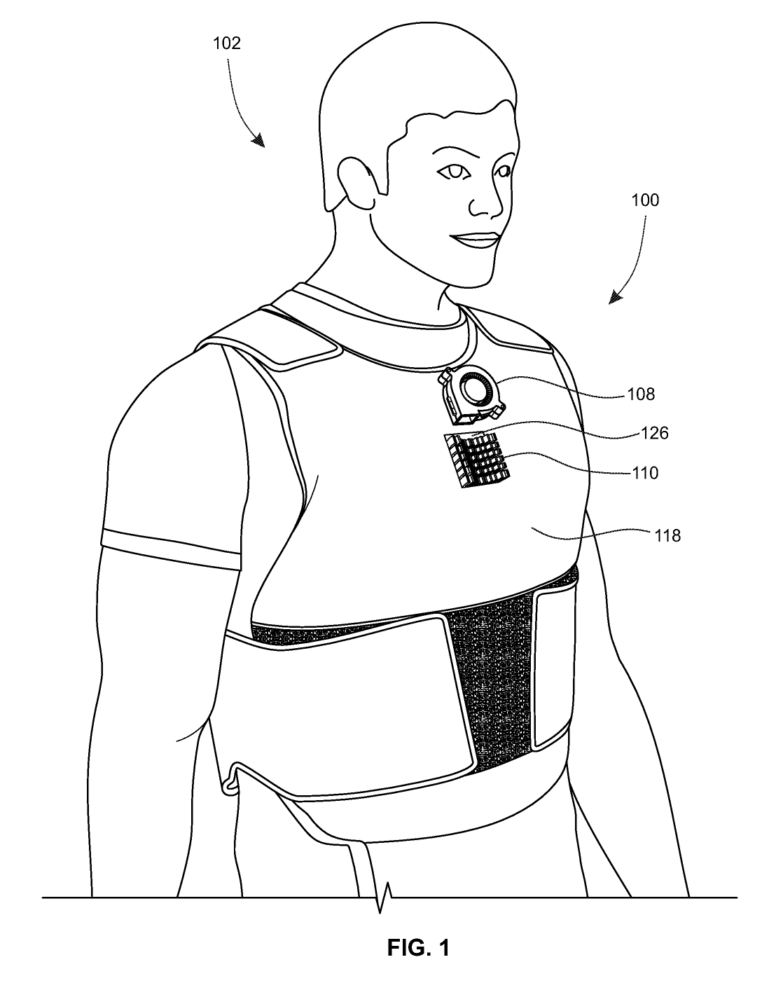

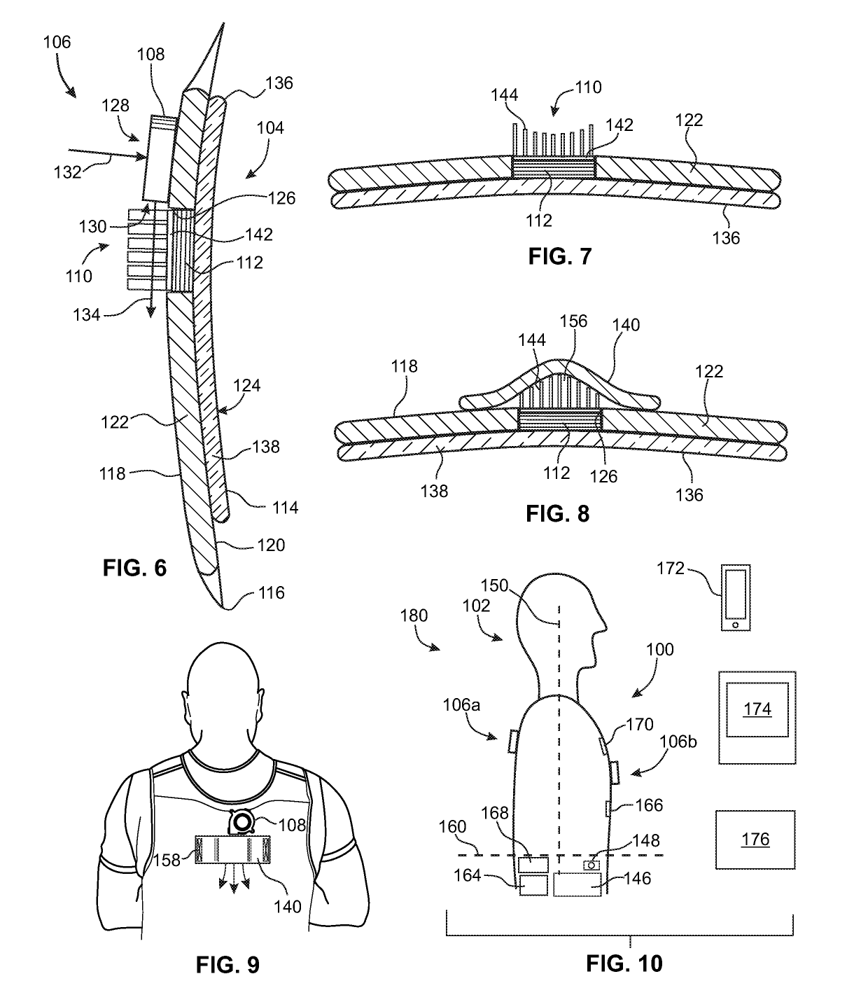

With reference to FIGS. 1 and 6, a preferred embodiment of a protective vest 100 may comprise a central vertical axis 150, a transverse axis 152, a sagittal axis 160, a vest shell 116, at least one cooling subsystem 104 and a power supply 146. The vest shell 116 may include an outer layer 118, an inner layer 120 and a ballistic armor layer 122 disposed therebetween. Each at least one cooling subsystem 104 may include a cooling disbursement element 114, a thermoelectric module 112 (e.g., one or more Peltier cooling devices) and a heat dissipation assembly 106. The cooling disbursement element 114 may comprise at least one coolant bladder 136 attached to the inner layer 120. The bladder 136 may include an external contact layer 124 of material which is significantly breathable, such as materials used in sport shirts. The heat dissipation assembly 106 may include a fan element 108 and a heat sink 110. The thermoelectric module 112 may be in heat-transferring communication between the cooling disbursement element 114 and the heat dissipation assembly 106. The fan element 108 may include a fan inlet 128, a fan outlet 130, and may preferably be configured to pull air in an inlet flow direction 132 and force a flow of air across the heat sink 110 in an outlet flow direction 134. The fan element may be attached to the vest shell 116 by various means, including one or more stitches 154, adhesive, placement in a respective vest pouch, or the like. The power supply 146 may be in electrical power-providing communication with the thermoelectric module 112 and the fan element 108.

With reference to FIG. 6, in certain preferred embodiments of a vest 100, the vest shell 116 may have a heat exchange aperture 126 extending from the inner layer 120 of the vest shell 116 through the outer layer 118. In such embodiments, at least a portion of either the heat sink 110 or the thermoelectric module 112, or the combined heat sink 110 and thermoelectric module 112, may protrude through the heat exchange aperture 126 such that at least fins 144 of the heat sink 110 extend outwardly of the outer layer 118.

In particular preferred embodiment of the protective vest 100, the fan element(s) 108 is/are attached to the vest shell 116 at, for example, the outer layer 118. The heat sink 110 may include a fin base 142 and plurality of fins 144 extending outward therefrom to respective fin tips. With reference to FIG. 7 for illustration, the fin tips may have having relative positions defining a concave shape. In one alternative, as illustrated for example in FIG. 8, the fin tips may have relative positions defining a convex shape.

With reference to FIGS. 8 and 9, the protective vest 100 may comprise an armor shroud element 140 secured to the vest shell 116 and extending across the heat sink 110 so as to define a heat sink air flow duct 156 and protectively envelop the heat exchange aperture 126. The armor shroud element 140 would also comprise a ballistic armor. This helps ensure that projectiles which would otherwise be able to enter the vest through the heat exchange aperture 126 may be stopped by the armor shroud element 140. Attachment of the armor shroud element 140 to the vest shell may be accomplished by various means, including stitching 158, adhesive, insertion into a vest pouch, other like.

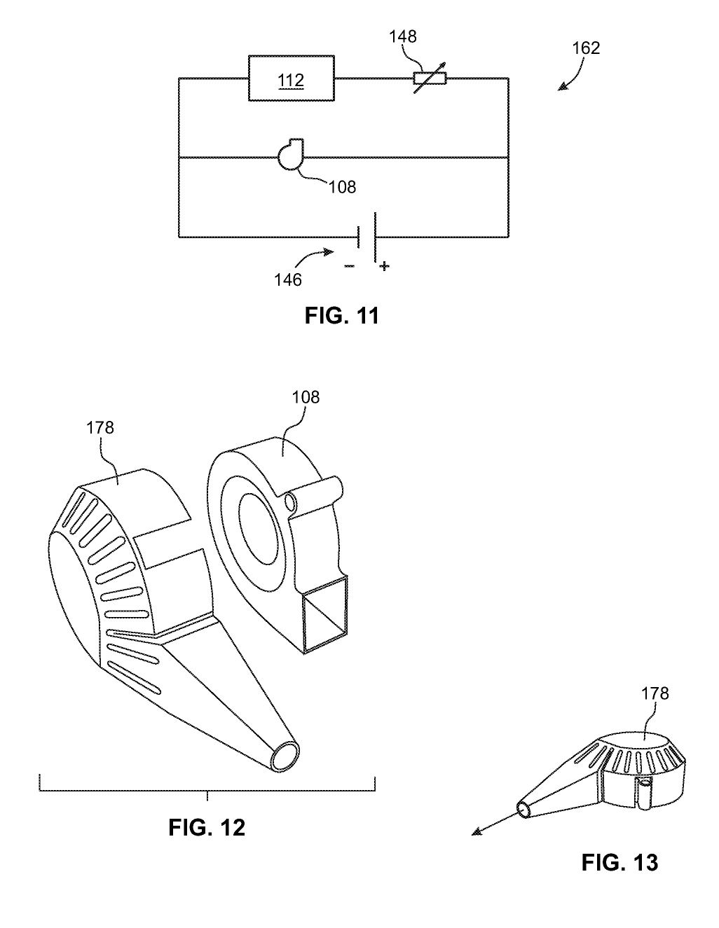

Certain preferred embodiments of a protective vest 100 may comprise a potentiometer element 148 for adjusting fan speed and thermoelectric module performance. This would allow the wearer 102, or an onboard computer system 168, to adjust and regulate the degree of cooling provided by the cooling subsystem(s) 104. FIG. 11 illustrates a schematic for one possible electrical circuit 162 including such a potentiometer element 148. An onboard computer system 168 may include, for example, a receiver element, a transmitter element, a processor element and a memory element.

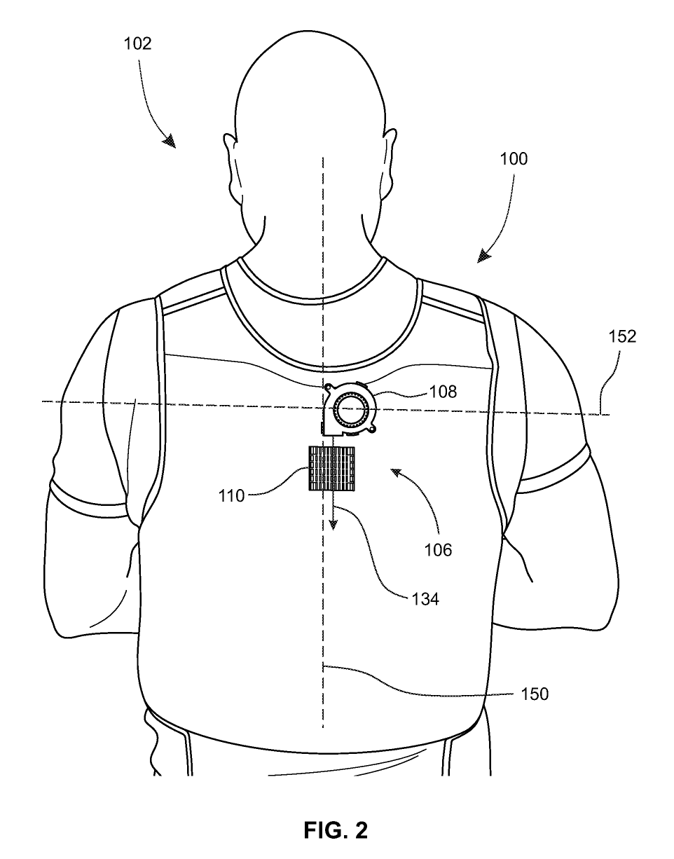

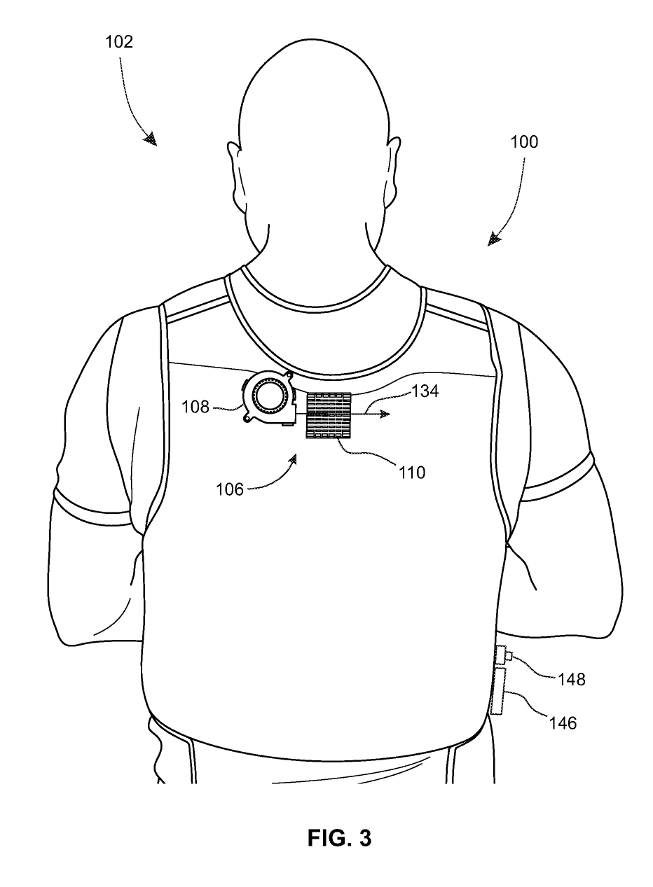

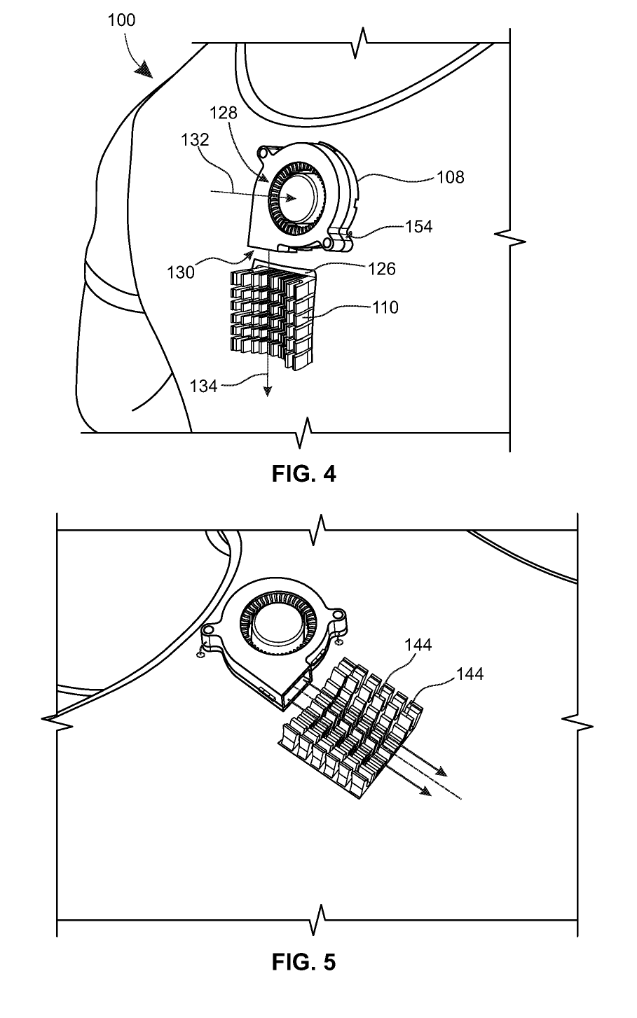

Referring to FIGS. 1, 2 and 10, the vest shell 116 may include a front portion and a rear portion. Moreover, the protective vest 100 may have an abdominal portion and a sagittal axis 160 defined extending through the abdominal portion. In such embodiments, the protective vest 100 may comprise at least two said cooling subsystems 104, one at the front portion and one at the rear portion. Additionally, the heat dissipation assembly 106a corresponding to the rear portion may be disposed at a first distance from the sagittal axis 160, and the heat dissipation assembly 106b corresponding to the front portion may be disposed at a second distance from the sagittal axis 160. The first distance may be greater than the second distance, thereby allowing the rear heat dissipation assembly 106b to avoid impingement by a seat back when the wearer is leaning against, for example, a vehicle seat. Additionally or in the alternative, with reference to FIG. 3, the fan element 108 and heat sink 110 may be oriented laterally to one another so as to avoid impingement by a seat back.

The coolant bladder 136 may contain a coolant 138, such as a coolant gel. It is preferable that the coolant is non-toxic to humans, in case a projectile passes through the vest armor and coolant bladder 136, and into the wearer 102.

With reference to FIG. 10, embodiments of the present invention may comprise a protective vest 100 controllable by a wearer software application. The wearer software application may comprise a program or group of programs. The program or group of programs may be particularly configured for simple and efficient operation by a wearer computing device 172 on which the application is installed (e.g., by a wearer of the corresponding protective vest). The wearer computing device 172 may be in data-receiving or data-sending communication with the protective vest 100 by way of an onboard computer element 168. A computing device may be or include, for example, a smart phone, tablet computer, vehicle-mounted computer, a combination thereof or the like. In particular embodiments, the computing device may be partially or completely securable within or to the vest.

With reference to FIGS. 1, 6 and 10, preferred embodiments of a protective vest system 180 may comprise a protective vest 100 and a wearer computing device 172. The vest 100 may include a vest shell 16, at least one cooling subsystem 104 and a power supply 146 in electrical power-providing communication with the thermoelectric module 112 and the fan element 108. The wearer computing device 172 may be in network communication with the protective vest 100 for sending vest commands to the protective vest and receiving vest information from the protective vest. The system 180 may further comprise a department computing device 174 in network communication with the wearer computing device 172. Moreover, the network communication between the department computing device 174 and the wearer computing device 172 may be way of a server element.

In particular embodiments of a protective vest system 180, the wearer computing device 172 may be configured to command (e.g., by way of a wearer software application) the speed of the fan elements 108 and the voltage applied to the thermoelectric modules 112. In certain embodiments of the system 180, the protective vest 100 may include a heart rate monitor 170 and at least one temperature sensor 166. In such embodiments, the wearer computing device 172 may be configured to receive (e.g., by way of the wearer software application) data from the protective vest 100 based on the measurements of the heart rate monitor 170 and temperature sensors 166.

In one preferred embodiment of a protective vest system 180, the protective vest 100 may include a GPS subsystem 164. In such embodiments, the wearer computing device 172 may receive data from the protective vest 100 based on positioning data of the GPS subsystem 164. The protective vest may include an onboard power supply 146, and the wearer computing device 172 may receive data from the protective vest 100 concerning the performance of the power supply 146 (e.g., current charge, capacity and the like).

A protective vest system 180 may comprise a plurality of protective vests 100 and a multiplicity of wearer computing devices 172. Each protective vest 100 may include, for example, a GPS subsystem 164 and a power supply 146 in electrical power-providing communication with the thermoelectric modules 112, the fan elements 108 and the GPS subsystem 164. The multiplicity of wearer computing devices 172 may each be in network communication with a respective one of the protective vests 100 for sending vest commands to the protective vest and receiving vest information from the protective vest. The vest information may include positioning data of the GPS subsystem 164. In such embodiments, each of the wearer computing devices 172 may present a map display indicating the geographical positions of each of the protective vests 100, for example, within a selected geographical location.

The protective vest 100 may be configured to communicate with the wearer computing device 172 via the wearer software application in a matter which allows the wearer computing device 172 to obtain information from the vest, and to transmit data which controls features of the vest 100. Embodiments of such a system may provide improved comfort and tactical advantage to the wearer by way of one or more features or capabilities, such as: (a) cooling the wearer's body temperature with miniature cooling fans, other cooling subsystems 104 or the like; (b) the placement or distribution of one or more temperature sensors 166 within the vest 100; (c) determining and transmitting the position of the wearer 102 by way of, for example, a GPS unit 164; measuring the vital signs of the wearer 102, such as heart rate via, for example, a heart rate monitor 170; receiving maintenance updates; data associating the wearer of the vest with certain employee information; and facilitating inbound and outbound notifications to/from the wearer, other wearers of similar protective vests, and operators of one or more department computing devices 174. Communications between the onboard computer element 168 or wearer computer device 172, and the wearer computer devices 172 of other wearers 102 or department computing devices 174, may preferably be by way of a server element 176

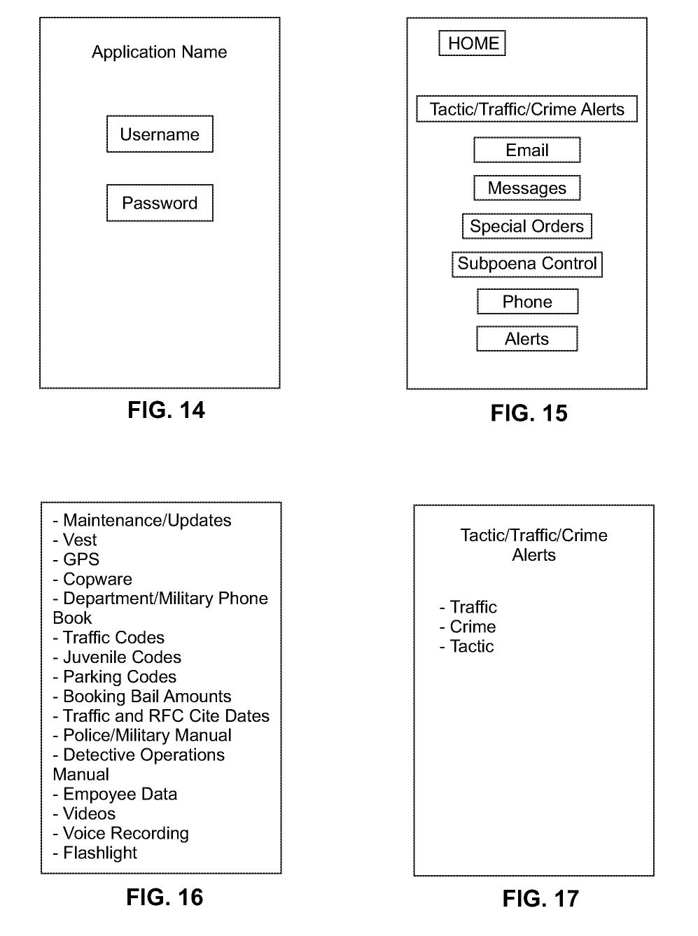

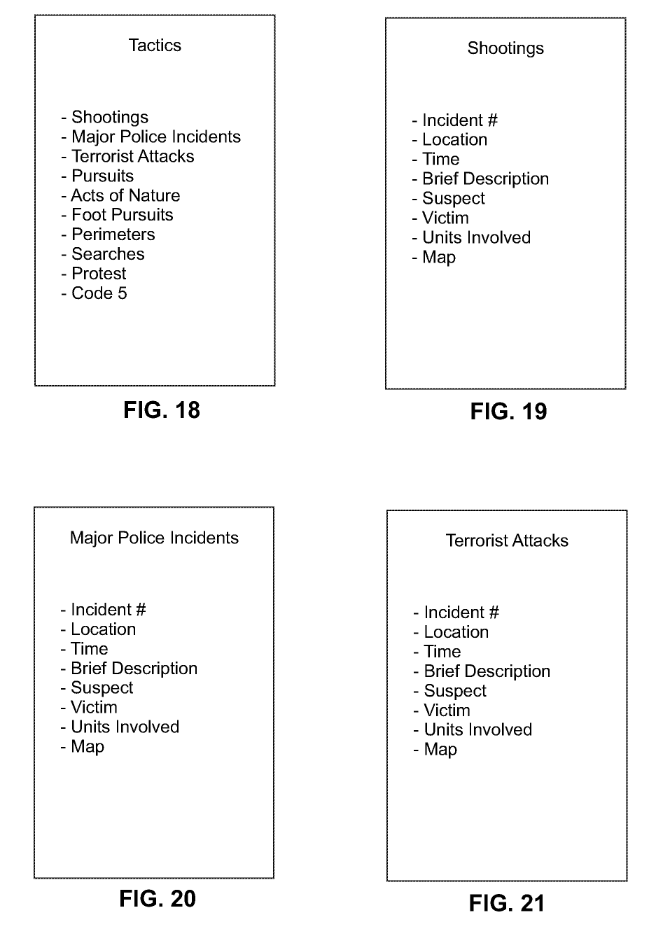

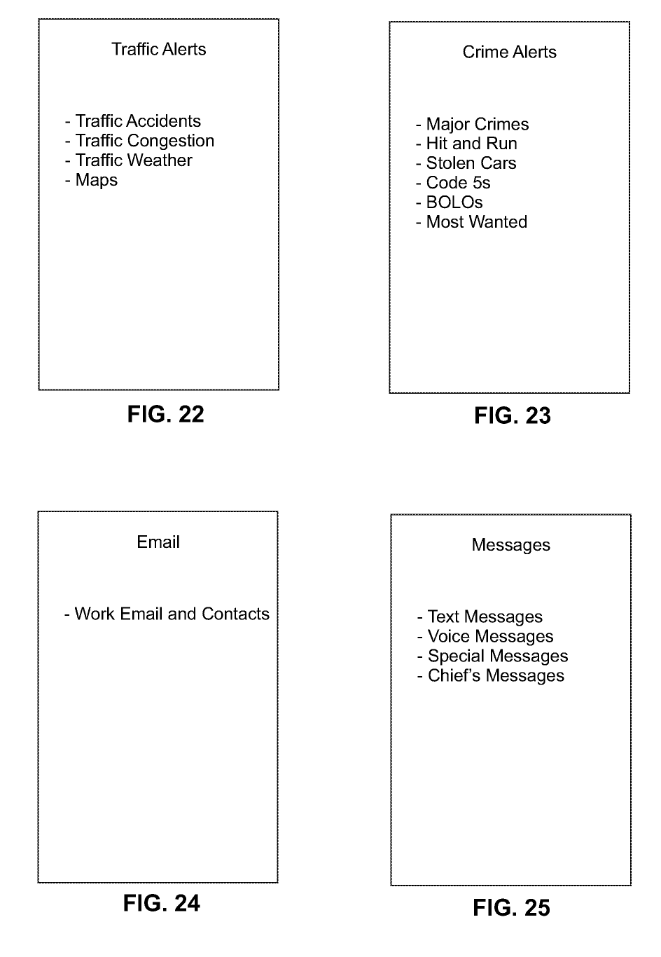

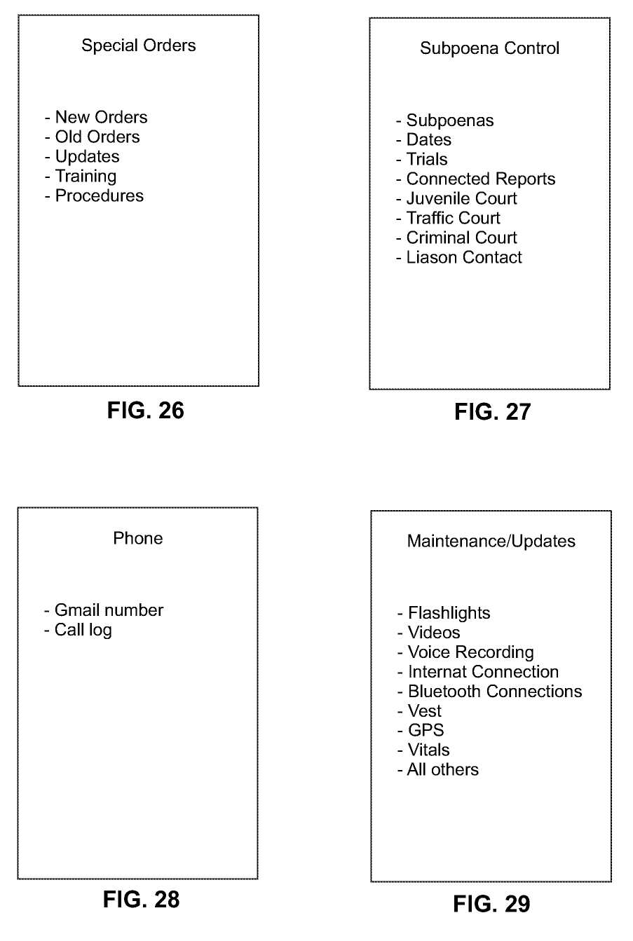

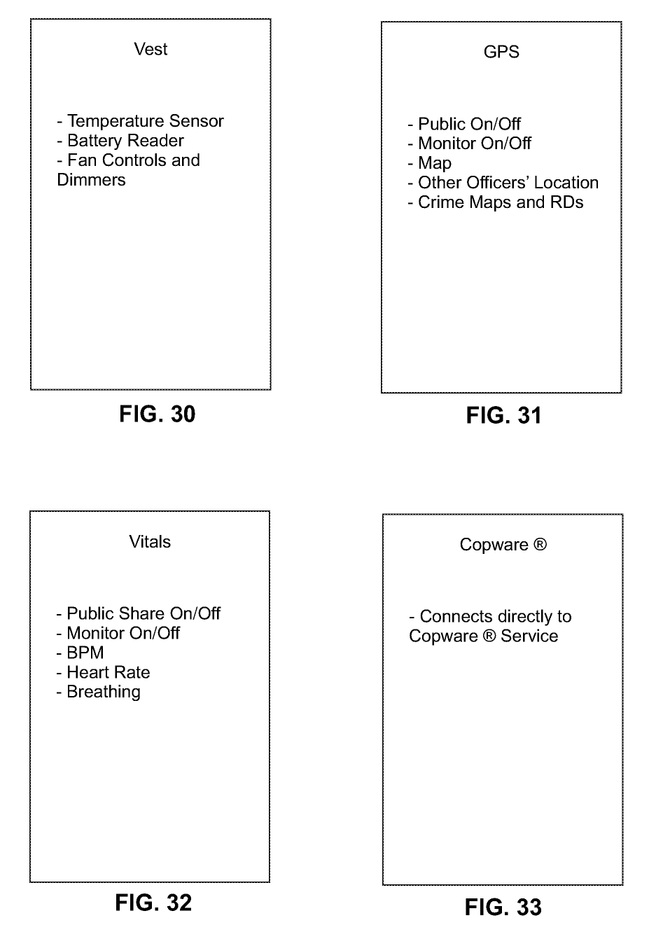

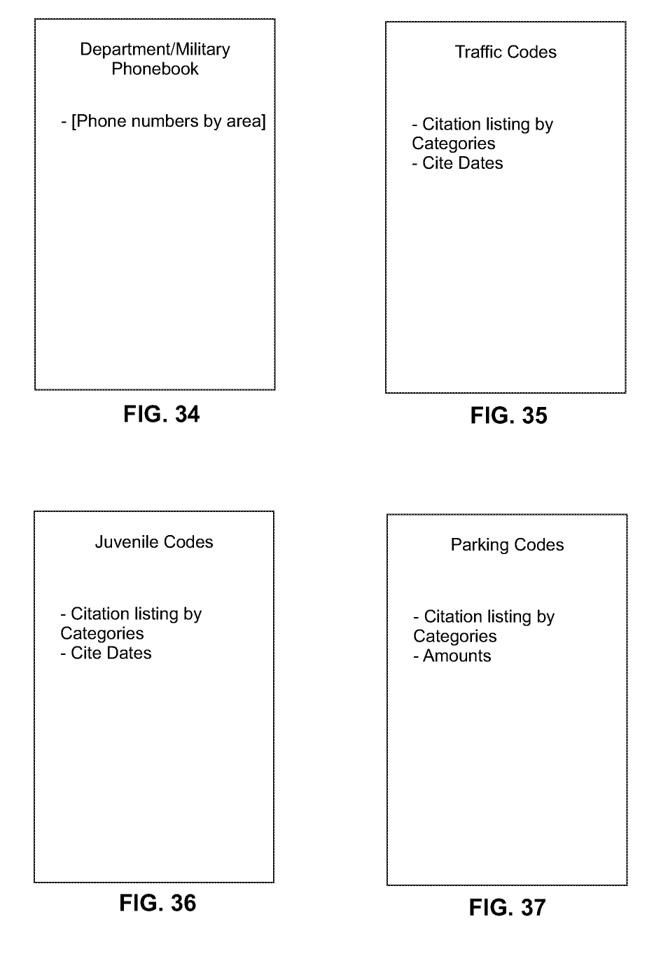

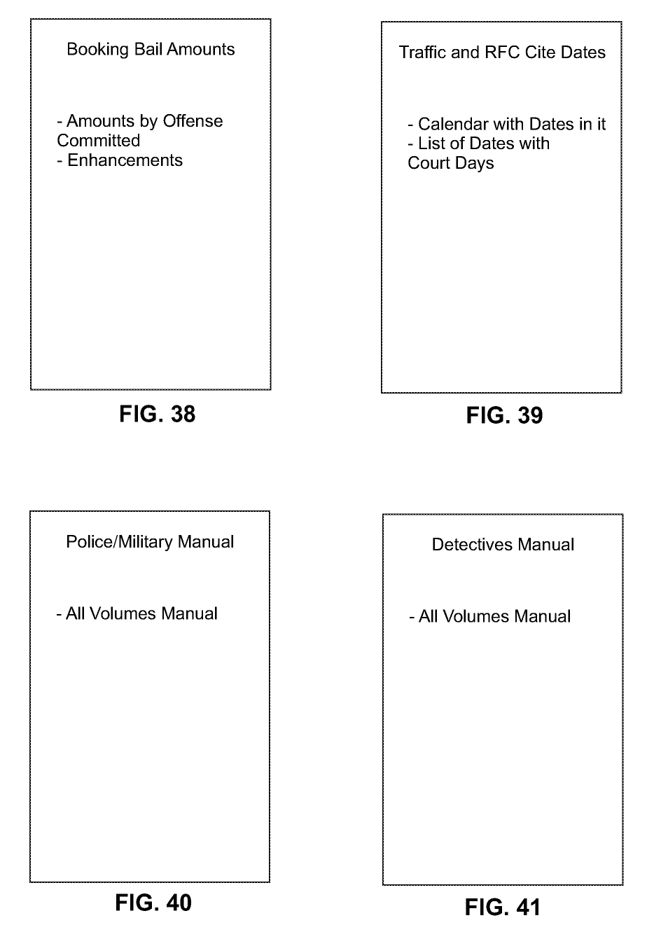

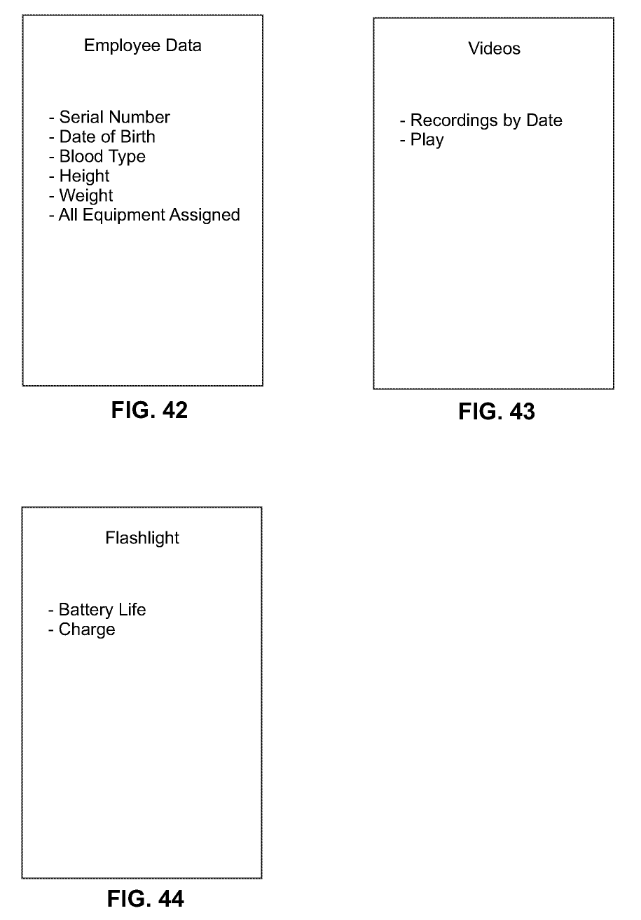

With reference to FIGS. 14-44, a wearer software application may be configured to operate on a wearer computing device 172. For example, in particular embodiments of the system described herein, the computing device 172 via the wearer software application may be configured to provide or run one or more of the following: Copware™ (conventional software for peace officers); traffic municipal code information; cite dates; booing bail amounts; juvenile municipal code information; release from custody information; municipal code sections; police department manual information; special orders; crime alerts; traffic alerts; and tactical alerts.

In preferred embodiments of a vest in accordance with the present invention, a cooling subsystem 104 is provided. The cooling subsystem is configured to cool the wearer of the vest. By way of example, the vest may have one or more fan elements 108 distributed about the vest to maximize cooling air flow at the optimal locations of the wearer's torso. For example, a particular vest may have up to 4, 12-Volt to 24-Volt miniature fans. The multiplicity of fans may be powered by a battery or multiple batteries (e.g., Lifebar battery by Antec, Inc. with 2 outputs of 5-Volts) and may be automatically controlled through the wearer software application or manually with an off and on switch, for example, on the battery 146. The cooling system 104 can also be used separately/independently (from all other functions) when used in the manual mode. In certain embodiments, the software application may control the fan speed of the fan elements 108, similarly to a dimmer switch, so that the system blows air at the wearer's comfort.

In particular preferred embodiments, the vest may also include one or more temperature sensors 166. Such temperature sensors may be place at or near the inner surface of the vest so as to come into close proximity the wearer's skin. These sensors may be positioned on the front portion of the vest, the rear portion of the vest, or both. Data from these temperature sensors may be received by the wearer's computing device 172 facilitated, for example, by the wearer software application. This temperature data may then be used to automatically adjust the speed of the fan elements 108 or thermoelectric modules 112 (in applicable embodiments) to help maintain the wearer's skin temperature at a pre-selected value. For example, the wearer 102 may select a particular skin temperature threshold setting which is most comfortable to him or her, and when the skin temperature rises above the pre-selected threshold value, the fan elements 108 may be commanded to turn on or increase fan speed, and thermoelectric elements 112 may be forced to produce a greater temperature differential.

The inner portion of the vest is in closest contact with the wearer's body. The cooling in this particular embodiment is preferably positioned for maximum cooling results. An aforementioned temperature sensor may optionally be placed below the fan element 108 or other cooling subsystem 104 (e.g., approximately 5 inches).

Cooling fans may also blow air through the rear portion of the vest, wherein the cooling fans are positioned for maximum cooling results. A temperature sensor may optionally be positioned below the upper rear fan (e.g., approximately 5 inches).

With reference to FIGS. 12 and 13, a shroud or fan housing 178 may be provided which displaces the spinning fan blades from the skin or shirt of the wearer. The shroud may include lateral slots, apertures or channels which allow air to flow into the fan blade section even though the outer rim or edge of the shroud is in contact with the shirt or skin of the wearer.

While the fan elements 108 may be configured with nozzles (such as by way of housing elements 178 for example) to direct the flow of air toward the locations on the vest or across heat sinks 110 most in need of cooling, it is envisioned that certain alternative embodiments of the vest the fans will include fan pouches or pockets which will house the respective fan elements 108, as well as direct their output air flow to particular locations along the inner surface of the vest. The fan pouches may be comprised of a mesh material so as to provide sufficient support for the fan, while allowing the forced air produced by the fan to flow through the pouch material. The housings may be comprised of silicon (for softer feeling in the vest), a more rigid polymer, or the like.

FIG. 13 depicts an embodiment of a fan element 108 including a funnel or nozzle at the output (the nozzle being provided by a housing 178 in this case). In certain embodiments, such nozzles may have a variety of different shapes and sizes, and may be removable and interchangeable at the option of the wearer. A USB-type connector plug is one type of connector which may be provided as a means of connecting a fan element 108 to a power source 146. For example, fan elements may include a one or two wires that are connected to the USB plug. The USB plug may have a miniature amplifier that amplifies, for example, 5-Volts to 12-Volts or 24-Volts.

A power supply 146 (e.g., a battery element) may be used to power fan elements 108, thermoelectric modules 112 and an onboard computer element 168 of certain embodiments of the present invention. For example, the USB plug of a fan element would plug into the battery element 146. In particular embodiments, the battery element or elements that are connected to the vest may be configured to communicate (e.g., via Bluetooth or the like) to the wearer software application. In such embodiments, the battery's performance may be monitored and controlled by the software application running on an associated wearer computing device 172.

In certain embodiments of a protective vest 100, a pouch element may be configured to receive and house a battery element. The pouch may act as a carrier that attaches to a utility belt of the wearer. In particular embodiments, two battery elements may fit into a single pouch for securement to a wearer's utility belt. The pouch may have openings in each side for USB plugs (or similar connectors) that plug into the battery elements. Once the plugs are connected to the respective batteries, the pouch may be mounted on the user in accordance with their preferences.

In particular elements of a system in accordance with the present invention, a GPS (Global Positioning System) subsystem 164 may be inserted into or otherwise connected to the protective vest 100. This may allow other officers (e.g., other wearers of respective protective vests 100) or supervisors to determine the real-time location of the wearer and therefore more efficiently and effectively coordinate a response to a major incident or tactical plan of approach (e.g., in the case of a home search, perimeter or major emergency). The GPS subsystem 164 may be controlled by the wearer (e.g., via on and off switch) by way of the wearer software application (e.g., via Bluetooth communication between the wearer computing device 172 and the vest 100). Moreover, the wearer would be able to see where other partner officers are and where the wearer themselves are.

In certain preferred embodiments, a vital strap 170 may be provided to sense and help monitor the real-time heartrate, breathing, and other parameters related to the wearer's health and performance. The vital strap may be inserted into the vest, and may form part of the actual vest that is securing the vest to the user. The vital strap may be adapted to communicate (e.g., via Bluetooth) with the wearer computing device by way of the wearer software application so that the wearer is able to monitor his/her own vitals. The wearer can also turn the application on for other officers and supervisors to view remotely during, for example, a tactical situation.

In certain preferred embodiments, some or all of the functions of the vest may be in communication with the wearer computing device 172 by way of the wearer software application described herein. Such functions may be intermittently or continuously monitored by the application for any maintenance required and/or emergency situations. By way of example, the wearer computing device (e.g., via software application) may be configured to continuously monitor or maintain one or more of the following items: (a) the operability and speed of the fan elements, including electrical wiring and connections; (b) the charge and performance of batteries connected to the fan elements and thermoelectric modules; (c) data from temperature sensors 166 distributed throughout the vest; (d) GPS location of the vest; (e) vital health parameters of the wearer; (f) quality and connectivity of the computing device's network (e.g., cell, satellite, wifi or internet) connection; and (g) Bluetooth communications with respective functions of the vest.

In particular embodiments of the protective vest system described herein, optional maintenance updates may be available if, for example, the company, police department, organization or user chooses to link the following to the wearer software application: (a) microphone status (audio records officer/wearer's interactions); (b) camera status (video records officer/wearer's interactions; and/or (c) flashlight status (e.g., communicated via Bluetooth)

Certain embodiments of the wearer software application described herein may also save or contain and transmit other information. For example, the vest may have a unique serial number (e.g., along with the size of the vest, expiration date, and/or make and model of the vest) that may be registered with the Company/Police Department/Organization/User that it belongs to. That information associated with the serial number of the vest, may have a date assigned to the wearer, the wearer's name, wearer's/employee's number and other personal information (such as, for example, date of birth, height, weight, blood type, and other possibly pertinent information).

The software application may support or run functions particularly useful for peace officers, including one or more of the following: (a) Copware™ (A law guide for law enforcement); (b) traffic municipal code information; (c) cite dates (court dates when issuing citations); (d) booking bail amounts (amounts used for the booking process); (e) juvenile municipal code information; (f) release from custody dates (known as RFC dates); (g) municipal code sections; (h) police department manual information; (i) special orders (new changes in policy); (j) crime alerts (pertaining to the city in which the vest is registered); (k) traffic alerts (in areas that the vest is in close proximity to); and (l) tactical alerts (when major police incidents, terrorist attacks, pursuits, acts of nature, shootings, etc. occur).

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A protective vest comprising:
a vest shell including an outer later, an inner layer and a ballistic armor layer disposed there between;
at least one cooling subsystem including a cooling disbursement element, a thermoelectric module and a heat dissipation assembly, the cooling disbursement element comprising at least one coolant bladder attached to the inner layer, the heat dissipation assembly including a fan element and a heat sink, the thermoelectric module being in heat-transferring communication between the cooling disbursement element and the heat dissipation assembly, the fan element being configured to force a flow of air across the heat sink; and
a power supply in electrical power-providing communication with the thermoelectric module and the fan element.

2. A protective vest as defined in claim 1 wherein
(i) the vest shell has a heat exchange aperture extending from the inner layer through the outer layer, and
(ii) at least a portion of either the heat sink or the thermoelectric module, or the combined heat sink and thermoelectric module, protrudes through the heat exchange aperture such that at least fins of the heat sink extend outwardly of the outer layer.

3. A protective vest as defined in claim 2 wherein the fan element is attached to the vest shell at the outer layer.

4. A protective vest as defined in claim 2 wherein the heat sink includes a fin base and plurality of fins extending outward therefrom to respective fin tips, the fin tips having relative positions defining a concave shape.

5. A protective vest as defined in claim 2 wherein the heat sink includes a fin base and plurality of fins extending outward therefrom to respective fin tips, the fin tips having relative positions defining a convex shape.

6. A protective vest as defined in claim 2 further comprising an armor shroud element secured to the vest shell and extending across the heat sink so as to define a heat sink air flow duct and protectively envelop the heat exchange aperture, the armor shroud element comprising a ballistic armor.

7. A protective vest as defined in claim 1 further comprising a potentiometer element for adjusting fan speed and thermoelectric module performance.

8. A protective vest as defined in claim 1 wherein
(i) the vest shell includes a front portion and a rear portion, and
(ii) the vest system comprising two said cooling subsystems, one at the front portion and one at the rear portion.

9. A protective vest as defined in claim 8 wherein
(i) the protective vest system has an abdominal portion and a sagittal axis defined extending through the abdominal portion; and
(i) the heat dissipation assembly corresponding to the rear portion is disposed at a first distance from the sagittal axis, and the heat dissipation assembly corresponding to the front portion is disposed at a second distance from the sagittal axis, the first distance being greater than the second distance.

10. A protective vest as defined in claim 1 wherein the coolant bladder contains a coolant gel.

11. A protective vest as defined in claim 10 wherein the coolant gel is non-toxic to humans.

12. A protective vest system comprising:
(a) a protective vest including
(i) a vest shell including an outer later, an inner layer and a ballistic armor layer disposed there between,
(ii) at least one cooling subsystem including a cooling disbursement element, a thermoelectric module and a heat dissipation assembly, the cooling disbursement element comprising at least one coolant bladder attached to the inner layer, the heat dissipation assembly including a fan element and a heat sink, the thermoelectric module being in heat-transferring communication between the cooling disbursement element and the heat dissipation assembly, the fan element being configured to force a flow of air across the heat sink, and
(iii) a power supply in electrical power-providing communication with the thermoelectric module and the fan element; and
(b) a wearer computing device in network communication with the protective vest for sending vest commands to the protective vest and receiving vest information from the protective vest.

13. A protective vest system as defined in claim 12 further comprising a department computing device in network communication with the wearer computing device.

14. A protective vest system as defined in claim 13 wherein the network communication between the department computing device and the wearer computing device is by way of a server element.

15. A protective vest system as defined in claim 12 wherein the wearer computing device commands the speed of the fan element and the voltage applied to the thermoelectric module.

16. A protective vest system as defined in claim 12 wherein
(a) the protective vest includes a heart rate monitor and at least one temperature sensor, and
(b) the wearer computing device receives data from the protective vest based on the measurements of the heart rate monitor and the at least one temperature sensor.

17. A protective vest system as defined in claim 12 wherein
(a) the protective vest includes a GPS subsystem, and
(b) the wearer computing device receives data from the protective vest based on positioning data of the GPS subsystem.

18. A protective vest system as defined in claim 12 wherein
(a) the protective vest includes a power supply, and
(b) the wearer computing device receives data from the protective vest concerning the performance of the power supply.

19. A protective vest system comprising:
(a) a plurality of protective vests, each protective vest including
(i) a vest shell including an outer later, an inner layer and a ballistic armor layer disposed there between,
(ii) at least one cooling subsystem including a cooling disbursement element, a thermoelectric module and a heat dissipation assembly, the cooling disbursement element comprising at least one coolant bladder attached to the inner layer, the heat dissipation assembly including a fan element and a heat sink, the thermoelectric module being in heat-transferring communication between the cooling disbursement element and the heat dissipation assembly, the fan element being configured to force a flow of air across the heat sink,
(iii) a GPS subsystem, and
(iv) a power supply in electrical power-providing communication with the thermoelectric module, the fan element and the GPS subsystem; and
(b) a multiplicity of wearer computing devices, each wearer computing device being in network communication with a respective one of the protective vests for sending vest commands to the protective vest and receiving vest information from the protective vest, the vest information including positioning data of the GPS subsystem.

20. A protective vest system as defined in claim 19 wherein each of the wearer computing devices presents a map display indicating the geographical positions of each of the protective vests within a selected geographical location.
